(19) United States
(12) Patent Application Publication
(10) Pub. No.: US 2014/0171753 A1
(43) Pub. Date: Jun. 19, 2014

(54) PORTABLE MEDICAL MONITORING SYSTEM WITH CLOUD CONNECTION AND GLOBAL ACCESS

(71) Applicants: Leo MONTEJO, Fremont, CA (US); Christian ILMI, San Jose, CA (US)

(72) Inventors: Leo MONTEJO, Fremont, CA (US); Christian ILMI, San Jose, CA (US)

(21) Appl. No.: 14/106,613

(22) Filed: Dec. 13, 2013

Related U.S. Application Data
(60) Provisional application No. 61/737,638, filed on Dec. 14, 2012.

(51) Int. Cl. A61B 5/00

(52) U.S. Cl. CPC A61B 5/0022; A61B 5/0004
USPC 600/301

(57) ABSTRACT

A portable measurement device configured to receive vital sign measurements and wirelessly transmit the vital sign measurements is presented. The transmitted vital sign measurements may be received, in real time, by a computing device that stores a list of patients and pre-programmed alert generation condition for each of the patients. When the vital sign measurements fulfill the alert generation condition, the computing device generates an alert, for example to medical staff tasked with the remote monitoring. One or more of EKG data, pulse oximetry, heart rate, pulse rate, systolic blood pressure, diastolic blood pressure, non-invasive blood pressure, and temperature may be monitored remotely using the inventive concepts.

### CROSS-REFERENCE TO RELATED APPLICATION(S)

This application claims the benefit of U.S. Provisional Application No. 61/737,638 filed on Dec. 14, 2012, the content of which is incorporated by reference herein.

### FIELD OF INVENTION

The disclosure relates generally to systems and methods for monitoring physical conditions and particularly to monitoring physical conditions remotely.

### BACKGROUND

Life expectancy in United States and the industrialized western world has risen dramatically over the past century. As a corollary to the increased life expectancy, the population is generally growing older and there is a rapid increase in the number of people with chronic medical conditions that need monitoring. One of such chronic conditions is heart ailment. According to some studies, about 64% of people over the age of 65 suffer from some form of chronic heart condition and/or hypertension. Among people who are 45 years and older, heart diseases far outnumber other causes of hospitalization.

Most patients with chronic conditions can function normally most of the time, but benefit from being monitored because they may be more likely to experience an emergency than someone who does not have a chronic condition. One obvious way to monitor the patient is by hospitalizing him, and connecting him to monitoring devices that are constantly viewed by trained medical staff. However, for various reasons, this is not a practical solution. Not only will people with chronic heart or other conditions be unhappy about being hospitalized for prolonged periods when they can function normally most of the time, it would be inefficient to have these patients take up hospital space and resources that should be used for patients with more acute conditions.

To address the situation, different devices have been introduced in the market. One of these devices is a portable alarm device that someone with a chronic condition can wear or carry, so that he can push a button when an emergency arises. These devices, however, are limited in their effectiveness because there is no medically trained party that is involved in the monitoring process—it is completely up to the patient to determine when he is having an emergency and to have the presence of mind to remember to push an alarm button. Furthermore, monitoring only the heart has limited usefulness since other conditions such as poor oxygenation can lead to a cardiac event. There are variations of such device available in the market today but the accuracy of monitoring falls short of what would be provided during hospitalization because monitoring is done by the patient himself.

Another type of device that is available is a portable recording device that a patient can wear, allowing his vitals to be taken and recorded continuously. A limitation of this type of device is that the recorded data is not reviewed by a trained medical personnel or a physician until it is accessed by them, for example by the patient taking the recording device in or taking some other measure to send the data to his physician. It could be days until the measurements are reviewed by a trained medical personnel. Hence, while this type of recording device may be helpful for diagnosing a condition, it is not effective for responding quickly to an emergency.

Yet another type of device connects to traditional monitors at hospitals and other care facilities and relays the information to a server, which allows users to view the data from distant locations. This type of device is limited in usefulness because interfaces to common medical devices are ever-changing and new devices crop up every day, all with different protocols, and adjustments would have to be made continually to the device.

Hence, today, hospitalization remains the most reliable way to monitor patients. A device that provides the benefits of hospitalization without actually requiring hospitalization is desirable.

### SUMMARY

In one aspect, the inventive concept pertains to a portable measurement device comprising at least one port configured to receive vital sign measurements, and a wireless transmission component configured to transmit the vital sign measurements.

In another aspect, the inventive concept pertains to a system for remotely monitoring a patient's vital signs. The system includes a portable measurement device configured to receive vital sign measurements and wirelessly transmit the vital sign measurements, and a computing device configured to receive the vital sign measurements in real-time and generate an alert if the vital sign measurements fulfill a predetermined condition. The system allows patients to be remotely monitored by trained medical staff.

In yet another aspect, the inventive concept pertains to a non-transitory computer-readable medium storing instructions that, when executed, cause a computer to perform a method for monitoring vital signs of a patient. The method entails receiving, in real time, vital signs from a plurality of remote measuring devices, the vital signs from each of the remote measuring devices including one or more of EKG data, pulse oximetry, heart rate, pulse rate, systolic blood pressure, diastolic blood pressure, non-invasive blood pressure, and temperature. The method further entails comparing the received vital signs with preprogrammed alert generation conditions, and generating an alert if the alert generation conditions are met by the received vital signs.

### BRIEF DESCRIPTION OF THE DRAWINGS

FIGS. 1A, 1B, 1C, and 1D each depicts a data acquisition device in accordance with one embodiment of the inventive concept.

FIG. 2 depicts a data acquisition device used with a network.

FIG. 3A and FIG. 3B each depicts an example of content that may be displayed on the local display of the data acquisition device and/or a monitoring terminal.

FIG. 4 depicts an example of a monitoring device at a back-end monitoring terminal.

FIG. 5 is a flowchart depicting the alert generation process in accordance with one embodiment of the inventive concept.

### DETAILED DESCRIPTION

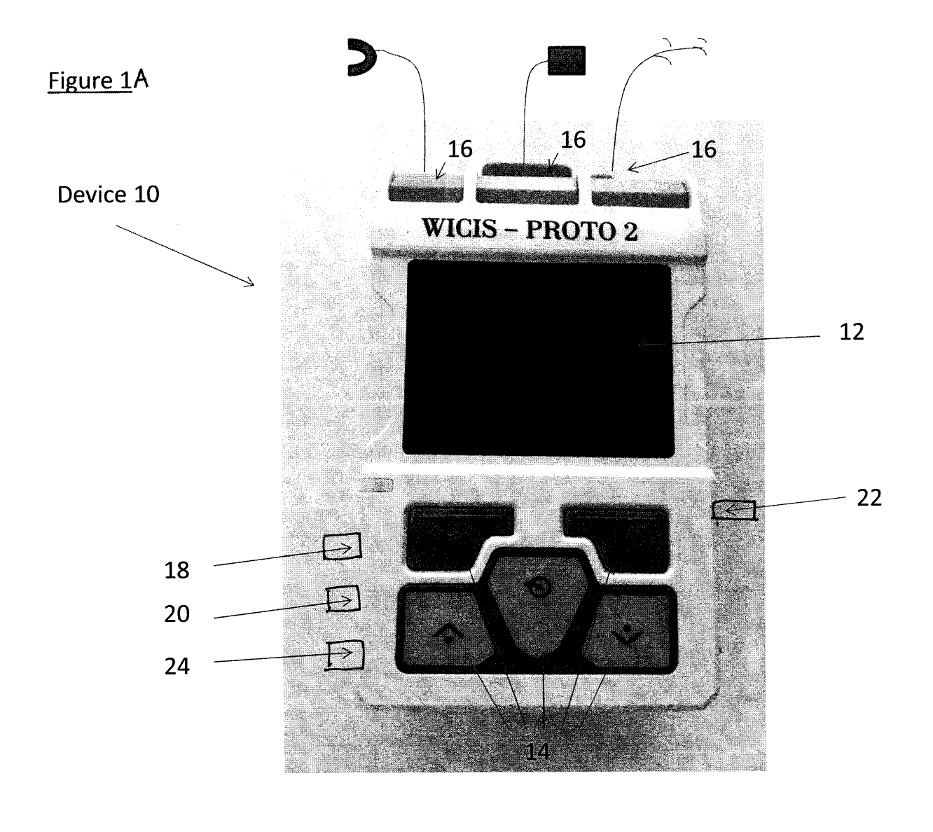

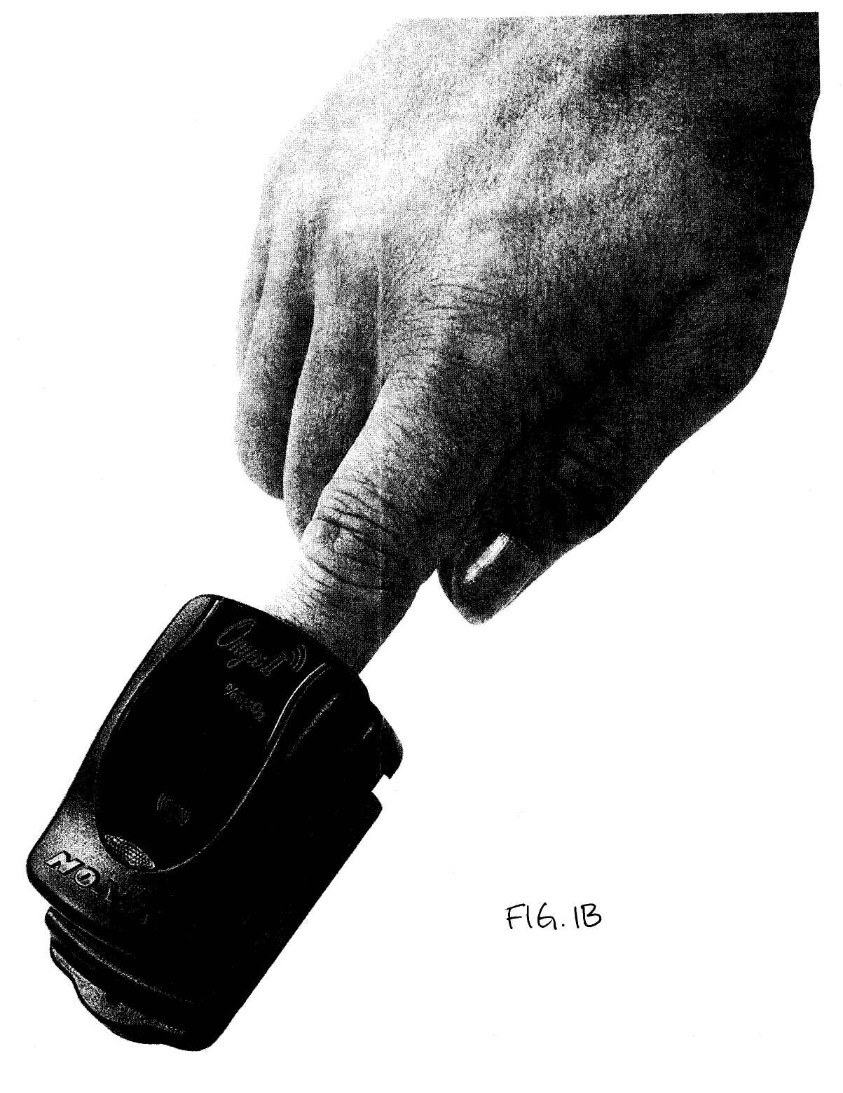

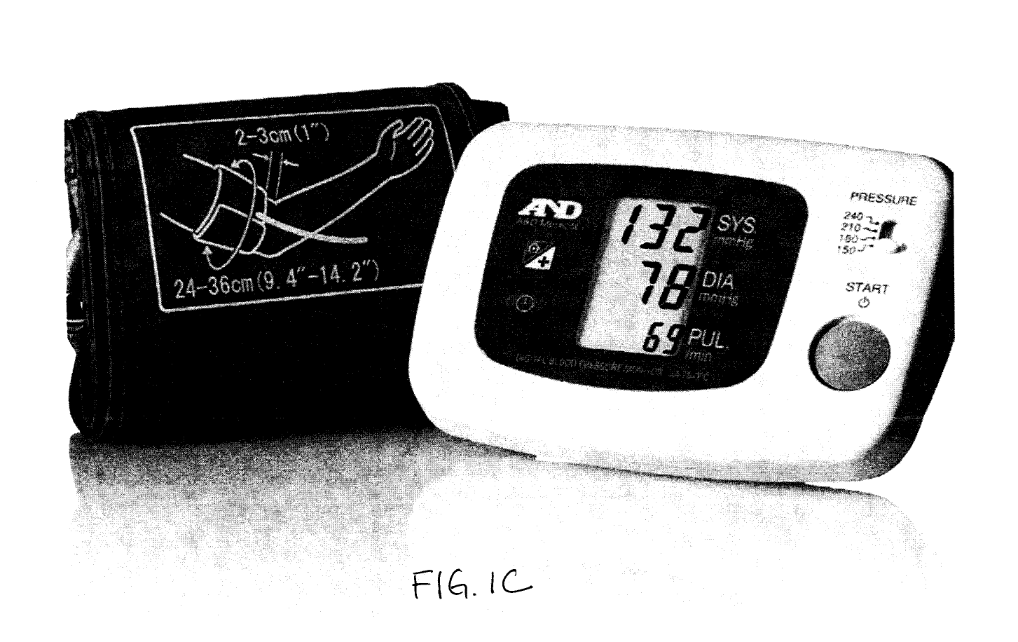

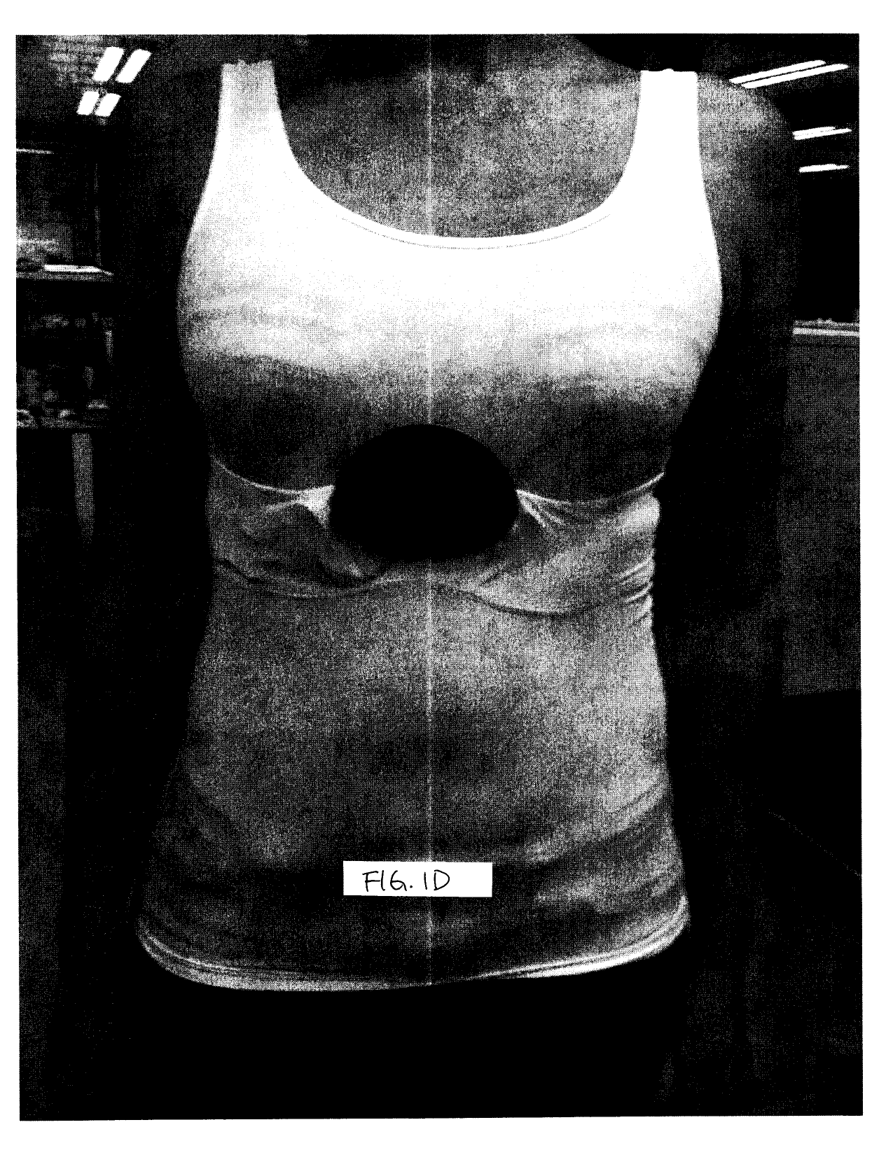

The data acquisition device that is disclosed herein allows patients to be monitored by medical personnel real-time from a remote location. For the purpose of simplifying the system description, we will focus on the device depicted in FIG. 1A. The other devices such as those depicted in FIGS. 1B, 1C, and 1D have substantially the same system functions as the embodiment shown in FIG. 1A. Sometimes, the data acquisition devices may only monitor a subset of the parameters that are explicitly described below or monitor different parameters. These parameters may be various vital signs that are measured using different probes.

The data that can be monitored include EKG waveforms, SpO2 waveforms, heart rate, SpO2, pulse rate, non-invasive blood pressure, and temperature. The data taken from a patient is uploaded to the cloud via WiFi (or a wireless/cellular network, or Bluetooth using an intermediate device such as a cell phone or a tablet) to become accessible by any computing device with a browser with Internet connection. The device, which is small enough to be worn by the patient wherever he goes, takes measurements from probes that are connected to the patient and transmits the measurement data to the cloud continually as long as a wireless connection is available. When the wireless connection is not available, the device has the capability to store the vitals until the connection is restored, then dump the data to the cloud. When the device does not have the capability to store the data during a loss of network communication, the intermediate device (the cell phone or the tablet) will do it through a mobile or Internet-based application. A person in charge of monitoring the patient can access the measurement data from any computer real-time, for example from a hospital or a physician's office. In addition to providing the real-time measurement data, the device generates alerts based on conditions that are predefined by a physician, so that prompt attention may be given in an emergency. Optionally, the device may be equipped with a GPS chip that transmits location information with the measurement data so that when an alert is generated, the person who is monitoring the patient will know the location of the patient.

The data acquisition device that is disclosed herein is a front-end device that is usable as a free-standing unit or with a server system and a backend monitoring unit. The data acquisition device is non-invasive, portable, and can be easily attached to a patient without significantly interfering with the patient's activities or movement. The probes connected to the data acquisition device may be existing probes that medical personnel are familiar with, although this is not a limitation of the inventive concept. Implementation with familiar probes makes training of the medical personnel more efficient. When used with a network, security measures are taken to preserve patient privacy and limit access to patient information. A backend monitoring unit accesses the data through the network, and can be used by medical personnel at hospitals to monitor patients remotely. The backend monitoring unit uses a software that is user-friendly with a display that is familiar to doctors and medical personnel.

The phrase “real-time,” as used herein, is intended to indicate a time lag of less than five seconds between the acquisition of data and retrieval of the data from a remote location. A “computing device,” as used herein, is intended to include any device with a processor and a memory and is capable of connecting to a network, including but not limited to desktops, laptops, tablets, and smartphones.

FIG. 1A depicts a data acquisition device 10 in accordance with one embodiment of the inventive concept. In this particular embodiment, the device 10 operates as a stand-alone device. As shown, the data acquisition device 10 has a local display 12, control keys 14, at least one connection port 16 (three are shown in the particular depiction), a wireless transmission chip 18, a power supply source 20, a removable data storage 22, and a USB port 24. The data acquisition device 10 receives measurements from a patient through the connection port 16, which is configured to connect to various types of probes to take measurements such as EKG data, pulse oximetry, heart rate, pulse rate, systolic blood pressure, diastolic blood pressure, non-invasive blood pressure, and temperature. Vital signs that are typically monitored during hospitalization may be measured with the device 10. The measurement data may be displayed on the local display 12 and also stored in the removable data storage 22, which may be a secure digital (SD) card. The USB port 24 allows other devices to be connected, including but not limited to infusion pumps, fluid delivery pumps, glucose meters, etc.

The connection port(s) 16 is (are) configured to communicate with probes attached to the patient, e.g. via wire connectors. In one embodiment, the probes may include EKG probes attached to the patient, a cuff around the patient's arm that automatically inflate at preset times, an oximetry probe and a temperature probe. The local display 12, which may be a liquid crystal display (LCD) or an organic light emitting diode (OLED) display, shows the real-time measurement data as well as historical data and alerts. Control keys 14 can be used to change the displayed content, for example between real-time monitoring data and historical data. The wireless transmission chip 18 would not be used when the device 10 is used as a free-standing unit unconnected to the network. The power supply 20 may be a connection port for connecting to an outlet and/or a battery compartment that is designed to hold a standard battery (e.g., lithium battery).

In one embodiment, the measurement data is stored on the removable data storage (e.g., SD card) 22 so that when a doctor or a medical personnel wants to review the patient's measurements, the information on the removable data storage 22 can be accessed not only via the device 10 but also by using any other computing device that can read the removable data storage 22. The amount of data that can be stored is limited only by the capacity of the data storage 22.

The removable data storage 22 makes the device 10 easily usable for multiple patients. For example, a hospital or care center could have multiple data storages cards, one for each patient. When one patient is discharged and the device 10 is to be used for another patient, the first patient's removable data storage 22 is removed and replaced with a new removable data storage 22. The storage device 22 may be switched each time the device 10 is used with a different patient. A returning patient can continue to use his old storage device 22, which enables him to directly connect to his historical data stored in the cloud.

FIGS. 1B, 1C, and 1D illustrate alternative embodiments of the device 10. The embodiment of FIG. 1B may be attached to a patient's finger, the embodiment of FIG. 1C connectable to an armband that is designed to wrap around a patient's arm, and the embodiment of FIG. 1D is coupled to a garment so that the patient can “wear” the device as part of the garment.

Some embodiments of the device and system use GUID (Universal identifier-tagged data) to protect patient privacy. In these embodiments, data moving across the Internet is not tagged with the patient's name and is therefore HIPAA compliant. Instead, at the time when a patient is given a device, the back-end server generates a unique identifier (GUID) that is passed to the device. The device transmits data with GUID tags. At the server end (e.g., at the monitoring terminal 50), the data is matched again to a patient name and to the person or group of care-givers that have the right to view this data.

In some embodiments, data is tagged with server time (ticks) and it is therefore possible to continuously display on the browser or viewing method the delay between when the data was generated and when the data is being displayed. This additional information allows the clinician to properly assess the acuity of any situation.

The adaptability of the device and system disclosed herein is enhanced by writing data to browsers such as Google Chrome, Safari, Internet Explorer, etc. A user would not need to download an app or obtain a specific software for the viewing device or the monitoring terminal. The viewer software would function independently of the operating system, being able to run without changes on Windows devices, Apple OS devices, Android devices, etc.

Due to the back-end server having a defined protocol, it can easily accept any current or future medical devices which transmit any type of medical data. The architecture will treat any new variable no differently than the device variables described above.

In some embodiments, the back-end server at the monitoring terminal 50 is built with the same tools that YouTube has been built with, and offers good scalability. This way, the system may grow easily to handle millions of concurrent users.

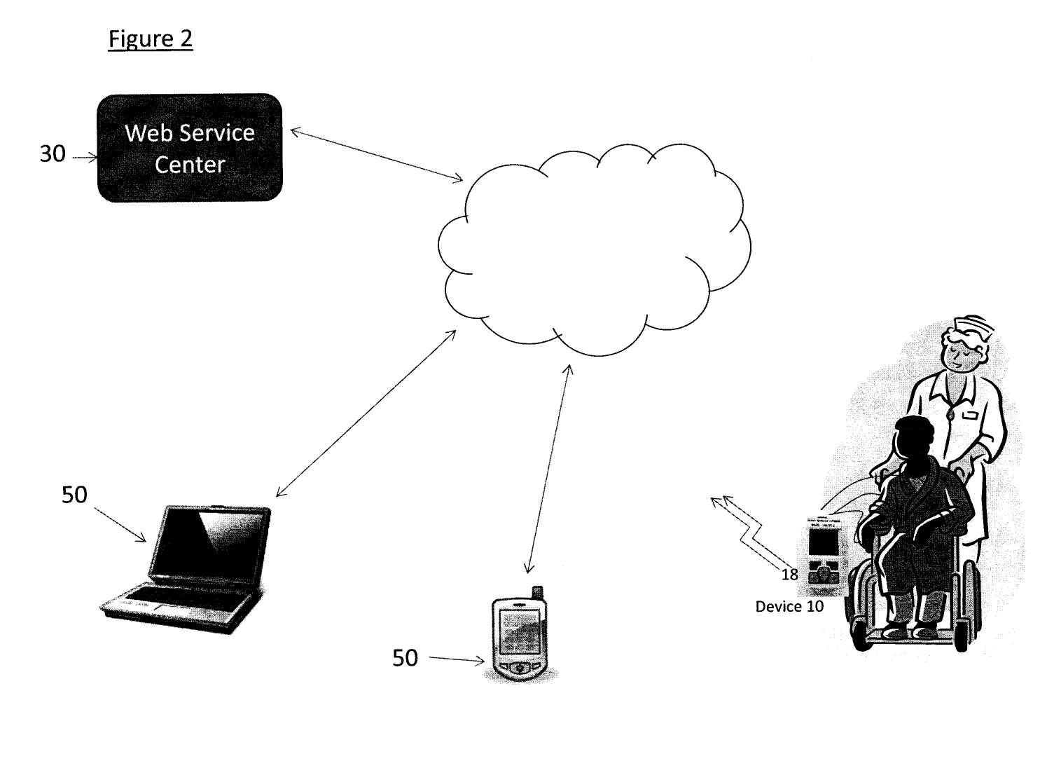

FIG. 2 shows the data acquisition device 10 used with a network. In this embodiment, the measurement data is transmitted to and stored in the cloud to be retrieved from any monitoring terminal 50 that has access to the network. A medical staff member at a doctor's office or hospital may monitor the patient's measurement data real-time by using a computing device at the backend monitoring terminal 50. The data acquisition device 10 reads data received from the connection port 16, displays that data on the device's own screen 12 and passes it on to the wireless transmission chip 18 in a proprietary format, in addition to storing it on the removable data storage 22. The wireless transmission chip 18 searches for a wireless network (e.g., WiFi) access and upon detecting a network, transmits data to the web service center 30. The web service center 30 stores and redirects the data to the cloud. A patient's current measurement data as well as historical data resides in the cloud, making them available from anywhere, without any limitation of a firewall or a physical location of a hospital setting for example.

If the device 10 is offline (e.g., because no wireless network is available), data is stored in the removable data storage 22 until network connection is re-established, at which point the stored data is uploaded to the cloud. The removable data storage 22 is tagged for a specific patient to allow the cloud to track which one of a plurality of devices 10 is monitoring which patient. Data includes wave forms for EKG and pulse oximetry, as well as heart rate, pulse rate, systolic and diastolic blood pressures, mean blood pressure, and temperature.

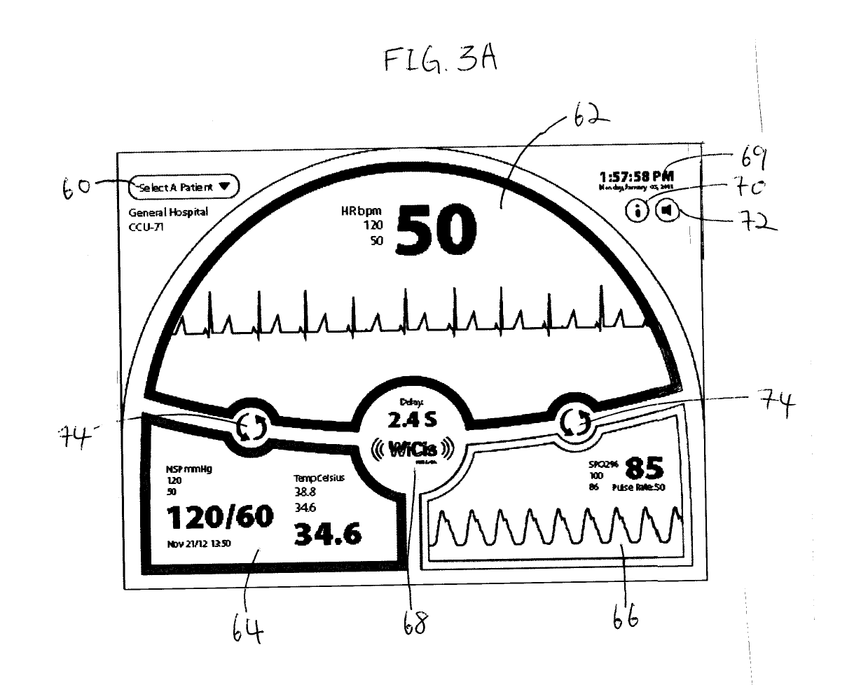

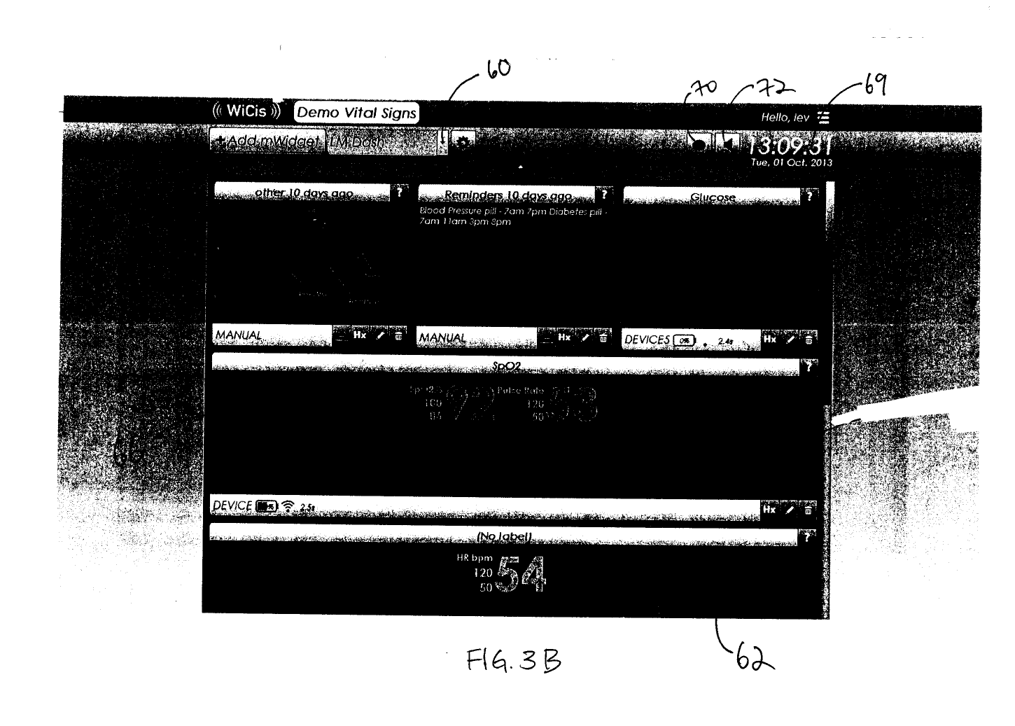

FIG. 3A depicts an example of content that may be displayed on the local display 12 of the data acquisition device 10 or the display unit of a monitoring terminal 50. A user goes through a log-in process to access the data that is shown. As shown, the user may select a patient by using a button 60. The display includes four general regions: EKG and Heart Rate window 62, Blood Pressure and Temperature window 64, Specific Oxygen window 66, and a Delay indicator 68 that shows how much time has passed since the measurement or data acquisition. This particular embodiment that is depicted in FIG. 3A shows the current time 69 and has other buttons for the user, such as a button for more information 70 and a button for turning on an alert 72.

The EKG and Heart Rate window 62 shows the EKG measurements moving across the screen as well as a heart rate number in beats per minute which, in the particular case that is shown, is 50. The Blood Pressure and Temperature window 64 shows the blood pressure measurement (120/60 in this particular example) and a body temperature in degrees Celsius (34.6° C. in this example). The Specific Oxygen window 66 shows a pulse moving across the window as well as a number indicating the oxygen level (it is 85% in this case) and the pulse rate taken from the SpO2 probe (50 in this case). The Delay indicator 68 shows that the data being displayed is 2.4 seconds old. The rotation buttons 74, when activated, move the relative positions of the Heart Rate window 62, the Blood Pressure and Temperature window 64, and the Specific Oxygen window 66 to allow the user to choose an arrangement that best suites his needs.

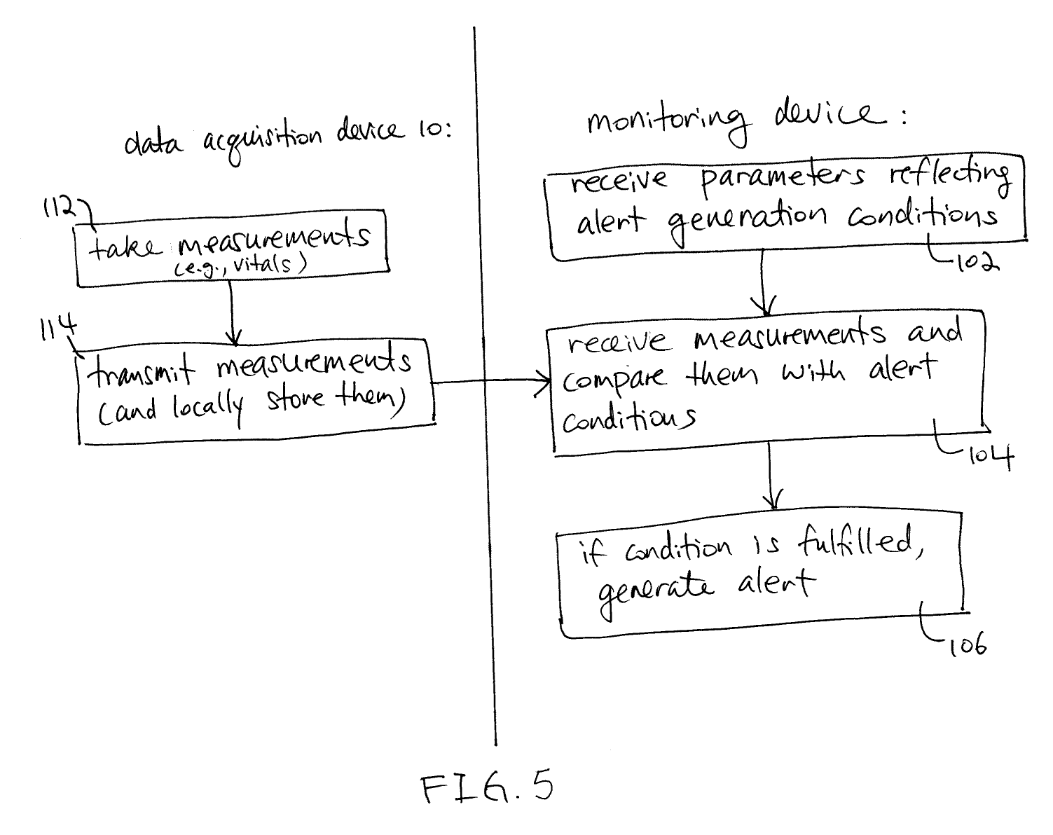

In some embodiments, the display of FIG. 3A may be color-coded so that a user can obtain the general state of the patient's condition with just a quick glance. As will be described in more detail in reference to FIG. 5 below, a computing device at the monitoring terminal 50 is pre-programmed with alert conditions. Hence, upon receiving a measurement, the computing device compares the measurement against alert conditions and determines what color code should be applied to display the measurement data. Alerting conditions will vary from patient to patient depending on the state of their health and any medical conditions they have. When the measurement is highly alerting (e.g., an emergency), the window showing that measurement may be highlighted or otherwise color-coded in red. A milder alert that encourages a more focused monitoring may be shown with an orange color code, and a normal reading may be shown with a yellow color code.

FIG. 3B shows another example of how content that may be displayed on the local display 12 of the data acquisition device 10 or the display unit of a monitoring terminal 50. As shown, substantially similar information is conveyed as in the example of FIG. 3A, but the windows are laid out differently. Also, additional windows showing the glucose level, reminders (e.g., reminders for tasks that are not yet completed), and an uploaded image are included in this example.

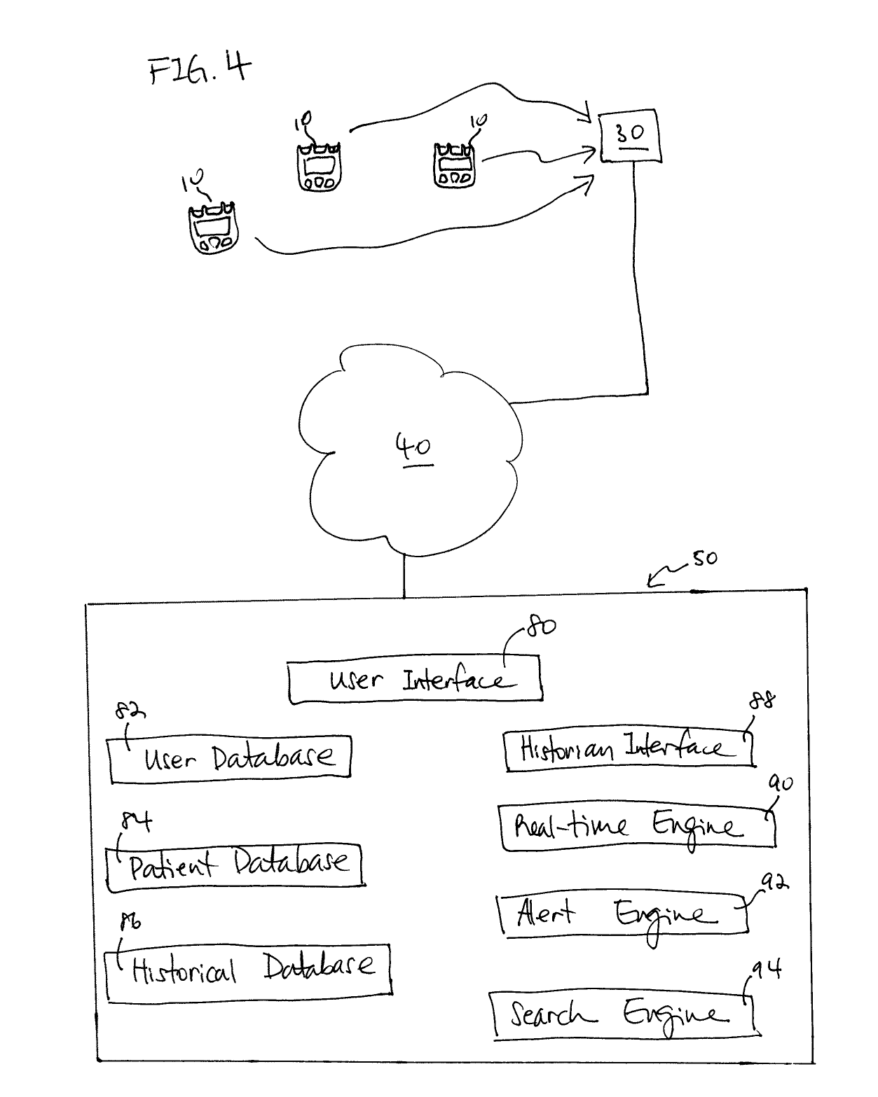

FIG. 4 depicts an example of a monitoring device at a back-end monitoring terminal 50. The monitoring device is a computing device that includes a user interface 80. The user interface 80 of the back-end monitoring device may be designed to automatically adjust to the screen size of the device being used, such as smartphones, tablets, or monitors. The user interface 80 allows a user to select the content of interest to be displayed (e.g., current measurement data, historical data, list of patients, list of users, diagnoses, alerts) and switch between them easily. The monitoring device includes a User Database 82, which is an internal database structure for managing users and their functions. The User Database 82 stores basic user information and the type of rights each user has. The User Database 82 may include login and password information for each user, as well as the type of user that is associated with each account. More specifically, different levels of access may be granted to different users.

The back-end server 30 further includes a Patient Database 84 that stores basic information (e.g., name, gender, birthdate, insurance) about each patient in the system. The back-end server is able to accommodate a SAAS infrastructure that allows for a large-scale web service and provide security services. There is also a Historical Database 86, which stores the historical data for each patient. Historical data may include past alerts. Several years of history may be stored in the cloud, and a portion of it or all of it may be downloaded onto the Historical Database 86 depending on the size of the memory available and the need. A third-party historian service may be used for storing real-time data that have been collected and made available to be retrieved in full fidelity. A Real-time Engine 90 receives data from the data acquisition device 10 and generates visualizations of the measurement data on the application in real-time. For example, waves and pulses would “move” across the user interface screen to reflect real-time measurements. An Alert engine 92 handles services for generating alerts, such as reviewing received measurements, comparing them against preset parameters, and generating signals for an alert. The alerts that are generated may be stored in the Historical Database 86. A search module 94 allows users to search stored information such as the Patient Database 84 and the Historical Database 86.

Users at the monitoring terminal 50 are granted different levels of access that are granted to different users of the monitoring device. In one embodiment, there are four levels of access that may be granted:
1. Administrators—users given this designation will have the ability to set up the system, add users, administer rights, create and edit diagnosis within allowed limits, perform simple device management, and see system alerts.
2. Admissions—users given this designation will have the ability to search for existing patients, edit patient information in a Patient Database 84, admit new patients by creating new patient accounts, and match patients with physicians.
3. Primary Care—users given this designation will have the ability to search for a patient, enter a diagnosis, edit the default values of the diagnosis, set up conditions for various levels of alert generation, choose a time frame for prescribing the device, and creating the prescription. Primary Care users will also have the full rights of a Viewer (see below).
4. Viewer—users given this designation will have the ability to search for patients, view charts, historical data, and live monitoring data, see patient alerts, and view basic patient information.

In some embodiments, all the levels of access have the rights of a Viewer. An administrator can easily change the level of access granted to each user on the list. Viewers or Primary Care users may have only the ability to see the patients they have been assigned. Unlike existing systems where any doctor logged into an Electronic Medical Record system (EMR) can see any patient's data on the EMR, the device and system disclosed herein allow a tighter control of the rights by limiting user access to certain patients. This feature further protects patient privacy.

FIG. 5 is a flowchart depicting the alert generation process 100. As shown, parameters based on conditions for alert are programmed into the monitoring device, perhaps by someone with a Primary Care level of access (step 102). The data acquisition device 10 continually collects measurements from a patient, which may include vital signs including but not limited to EKG waveforms, SpO2 waveforms, heart rate, SpO2, pulse rate, non-invasive blood pressure, and temperature (step 112), and transmits them to the computing device (step 114). Upon receiving the measurement data, the Alert Engine 92 of the computing device compares the measurements with the programmed parameters (step 104). When a condition is fulfilled, an alert is generated (step 106). Alerts can be implemented in a Web-based form such as an automatic email, text message, and/or an audiovisual signal output at the monitoring terminal 50. In response to the alert, the medical staff at the monitoring device can dispatch an ambulance or get in contact with a designated caretaker of the patient so that the patient can be brought in to the hospital/clinic.

A doctor or Physician may enter different types of Diagnoses for a particular patient. Entries may be received by the computing device in any suitable form and interface, such as a page with drop-down menus and values to be entered. A screen under “Diagnoses” will shows various measured conditions and a diagnosis.

The portable data acquisition device 10 frees the patient so that he can go about his ordinary activities without being hospitalized, while still benefiting from the monitoring done by trained medical staff. By uploading the measurement data to a cloud, the physicians and the medical staff are afforded more flexibility as well because the patient can be monitored from any computing device.

Although this disclosure is provided in the context of monitoring vitals, the general concept disclosed herein may be applied to monitoring different physiological parameters. Application of the disclosure may further be extended to monitoring various non-physiological parameters by using different types of probes or sensors. For example, the patient's location can be determined using the GPS of the tablet or the cell phone carried by the patient. The patient's movements can be determined using the accelerometers integrated in a garment worn by the patient or integrated in the tablet or the cell phone carried by the patient. The picture of the patient's wound can be uploaded along with his vitals to enable the monitoring physician make a judgment on the evolution of the healing.

Various embodiments of the back-end monitoring terminal may be implemented in or involve one or more computing devices. The computing device is not intended to suggest any limitation as to scope of use or functionality of described embodiments. The computing device includes at least one processing unit and memory. The processing unit executes computer-executable instructions and may be a real or a virtual processor. The computing device may include a multi-processing system which includes multiple processing units for executing computer-executable instructions to increase processing power. The memory may be volatile memory (e.g., registers, cache, random access memory (RAM)), non-volatile memory (e.g., read only memory (ROM), electrically erasable programmable read only memory (EEPROM), flash memory, etc.), or combination thereof. In an embodiment of the inventive concept, the memory may store software for implementing various embodiments of the present disclosure.

Further, the computing device may include components such as storage, one or more input computing devices, one or more output computing devices, and one or more communication connections. The storage may be removable or non-removable, and includes magnetic disks, magnetic tapes or cassettes, compact disc-read only memories (CD-ROMs), compact disc rewritables (CD-RWs), digital video discs (DVDs), USB memory sticks, Solid State Memory Devices or any other medium which may be used to store information and which may be accessed within the computing device. In various embodiments, the storage may store instructions for the software implementing various embodiments of the present disclosure. The input computing device(s) may be a touch input computing device such as a keyboard, mouse, pen, trackball, touch screen, or game controller, a voice input computing device, a scanning computing device, a digital camera, or another computing device that provides input to the computer system. The output computing device(s) may be a display, printer, speaker, or another computing device that provides output from the computer system. The communication connection(s) enable communication over a communication medium to another computer system. The communication medium conveys information such as computer-executable instructions, audio or video information, or other data in a modulated data signal. A modulated data signal is a signal that has one or more of its characteristics set or changed in such a manner as to encode information in the signal. By way of example, and not limitation, communication media includes wired or wireless techniques implemented with an electrical, optical, RF, infrared, acoustic, or other carrier. In addition, an interconnection mechanism such as a bus, controller, or network may interconnect the various components of the computer system. In various embodiments of the inventive concept, operating system software may provide an operating environment for software's executing in the computer system, and may coordinate activities of the components of the computer system.

Some embodiments of the inventive concept may be described in the general context of computer-readable media. Computer-readable media are any available media that may be accessed within a computer system. By way of example, and not limitation, within the computer system, computer-readable media include memory, storage, communication media, and combinations thereof.

Having described and illustrated the principles of the inventive concept with reference to described embodiments, it will be recognized that the described embodiments may be modified in arrangement and detail without departing from such principles. It should be understood that the programs, processes, or methods described herein are not related or limited to any particular type of computing environment, unless indicated otherwise. Various types of general purpose or specialized computing environments may be used with or perform operations in accordance with the teachings described herein. Elements of the described embodiments shown in software may be implemented in hardware and vice versa.

While the exemplary embodiments of the inventive concept are described and illustrated herein, it will be appreciated that they are merely illustrative. For example, while the disclosure focuses on monitoring vital signs, the inventive concept may be extended to monitoring of other physical conditions.

What is claimed is:

1. A portable measurement device comprising:
at least one port configured to receive vital sign measurements; and
a wireless transmission component configured to transmit the vital sign measurements.

2. The portable measurement device of claim 1, wherein the port is configured to communicate with probes that are capable of reading vital signs.

3. The portable measurement device of claim 1, further comprising a removable memory card configured to store the vital sign measurements.

4. The portable measurement device of claim 1, wherein the vital sign measurements comprise at least one of the following:
EKG data;
pulse oximetry;
heart rate;
pulse rate;
systolic blood pressure;
diastolic blood pressure;
non-invasive blood pressure; and
temperature.

5. The portable measurement device of claim 1 further comprising a display for visually communicating the vital sign measurements in real-time.

6. The portable measurement device of claim 5, wherein the display is capable of displaying historical data including past measurements.

7. The portable measurement device of claim 5, wherein the display is capable of simultaneously displaying real-time measurements for two or more of the following:
EKG data;
pulse oximetry;
heart rate;
pulse rate;
systolic blood pressure;
diastolic blood pressure;
non-invasive blood pressure; and
temperature.

8. The portable measurement device of claim 1, wherein the display is configured to generate an alert if some of the vital sign measurements fulfills a predetermined condition.

9. The portable measurement device of claim 1 further comprising a location identifier component generating location information of where the vital sign measurements were taken.

10. A system for remotely monitoring a patient's vital signs, the system comprising:
a portable measurement device configured to receive vital sign measurements and wirelessly transmit the vital sign measurements; and
a computing device configured to receive the vital sign measurements in real-time and generate an alert if the vital sign measurements fulfill a predetermined condition.

11. The system of claim 10, wherein the computing system is configured with a network connectivity to selectively download preselected data from a remote data storage.

12. The system of claim 10, wherein the portable measurement device is a first portable measurement device and the vital sign measurements are a first set of vital sign measurements, further comprising a second portable measurement device from which the computing system is configured to receive a second set of vital sign measurements.

13. The system of claim 12, wherein the first set of vital sign measurements and the second set of vital sign measurements are associated with different patient identifiers.

14. The system of claim 10 further comprising:
a user database storing a list of users and access level granted to each of the users; and
a patient database storing information about patients.

15. The system of claim 14 further comprising a historical database storing past alerts generated for each patient.

16. The system of claim 14, wherein the patient database stores programmed alert parameters for each of the patients, further comprising an alert engine configured to generate an alert if the programmed parameters are fulfilled for a patient.

17. A non-transitory computer-readable medium storing instructions that, when executed, cause a computer to perform a method for monitoring vital signs of a patient, the method comprising:
receiving, in real time, vital signs from a plurality of remote measuring devices, the vital signs from each of the remote measuring devices including one or more of the following:
EKG data;
pulse oximetry;
heart rate;
pulse rate;
systolic blood pressure;
diastolic blood pressure;
non-invasive blood pressure; and
temperature;
comparing the received vital signs with preprogrammed alert generation conditions;
generating an alert if the alert generation conditions are met by the received vital signs.

18. The computer-readable medium of claim 17 further comprising receiving location information of where vital signs were measured.

19. The computer-readable medium of claim 16, further comprising selectively downloading historical data from a remote storage.

20. The computer-readable medium of claim 17 further comprising:
receiving a login information identifying a user; and
checking access level granted for the identified user to determine type of operation allowed for the user.
